(19) United States
(12) Patent Application Publication
(10) Pub. No.: US 2021/0290402 A1
(43) Pub. Date: Sep. 23, 2021

(54) Semi-Radial Head Prosthesis

(71) Applicant: Ningbo No. 6 Hospital, Ningbo (CN)

(72) Inventors: Jianming Chen, Ningbo (CN); Guanyi Liu, Ningbo (CN); Guonong He, Ningbo (CN)

(73) Assignee: Ningbo No. 6 Hospital

(21) Appl. No.: 17/248,323

(22) Filed: Jan. 20, 2021

(30) Foreign Application Priority Data
Mar. 20, 2020 (CN) 202010199541.7

(51) Int. Cl. A61F 2/38

(52) U.S. Cl. CPC A61F 2/3804; A61F 2002/30433; A61F 2002/3827

(57) ABSTRACT

The present invention discloses a semi-radial head prosthesis for matching to a non-fractured part of a bone. The prosthesis comprises a prosthesis head (3) matching to a radial head of the non-fractured part, the prosthesis head having a fractured surface, the prosthesis head (3) is jointed with the non-fractured part (2) to form an intact radial head. In the present invention, when the partial articular surface of the radial head is fractured, it is unnecessary to remove the entire radial head. Instead, the radial head of the fractured part is replaced with a prosthesis head and the radial head of the non-fractured part is retained. The two parties are combined to form an intact radial head. This minimizes iatrogenic injuries to the patients, and is good for the joint intactness and joint stability.

### TECHNICAL FIELD OF THE INVENTION

The present invention relates to the field of medical instruments, in particular to a semi-radial head prosthesis.

### BACKGROUND OF THE INVENTION

The human elbow joint comprises the distal humerus, the proximal ulna and the radial head. The radial head fracture accounts for about 5.4% of all fractures in the human body. At present, the most commonly used clinical classification of radial head fractures is the Mason classification, comprising: type-I fractures, i.e., radial head fractures without displacement; type-II fractures, i.e., radial head fractures with partial joint displacement (comprising simple and comminuted partial joint fractures); type-III fractures, i.e., radial head fractures with completely comminuted joint. Radial head joint replacement has been proved to be effective in the treatment of type-II and type-III comminuted radial head fractures, with satisfactory mid- to long-term clinical results. The indications for surgery are provided mainly by comminuted partial and complete joint fractures of the radial head, especially when there are more than 3 fracture fragments. The surgical method is to expose the radial head by lateral incision, carry out radial head osteotomy by using a micro oscillating saw at the neck of the radius, ream the proximal medullary cavity of the radius to complete the anatomical matching of the prosthesis, and then insert the prosthesis trial model. Upon confirming the size of the radial head prosthesis, prosthesis of an appropriate size is selected.

However, radial head replacement is generally suitable for patients with: 1. the fracture of the entire articular surface of the radial head, as shown in FIG. 1; and 2. the fracture of the partial articular surface of the radial head, as shown in FIG. 2. However, at present, the radial head prosthesis used in clinical radial head replacement is an entire radial head. The entire radial head needs to be removed before the prosthesis is installed. It is suitable for the fracture of the entire articular surface of the radial head, not for the fracture of the partial articular surface of the radial head. Meanwhile, it is not uncommon for the fracture of the partial articular surface of the radial head in clinical practice, and the fracture of the partial articular surface of the radial head is often the fracture of half (about 50%) of the articular surface of the radial head.

At present, since there is no semi-radial head prosthesis, during the radial head replacement for the fracture of the partial articular surface of the radial head, it is necessary to remove the entire radial head, that is to say, comprising the fractured radial head part and the non-fractured radial head part. For patients with partial radial head joint fracture, it is necessary to remove the non-fractured part, which causes additional iatrogenic injuries to the patients and has the risks of joint instability and loosening of the radial head prosthesis after the operation.

### SUMMARY OF THE INVENTION

It is an object of the present invention to provide a semi-radial head prosthesis suitable for the fracture of the partial articular surface of the radial head in view of the prior art.

For achieving the above object, the semi-radial head prosthesis comprises: a prosthesis head matching to a radial head of the non-fractured part, the prosthesis head having a fractured surface, the prosthesis head is jointed with the non-fractured part to form an intact radial head.

The size and specification of the prosthesis head are designed according to the fracture range of the patient's radial head. Preferably, the volume of the prosthesis head is ¼ to ½ of the volume of the intact radial head. Thus, the prosthesis head and the non-fractured radial head part form a stable combination structure. Preferably, the prosthesis head is ½ of the volume of the intact radial head. The fracture of half (about 50%) of the articular surface of the radial head is common in clinical practice. The design of the size of the prosthesis head to be ½ of the intact radial head can improve the versatility of the prosthesis head.

Preferably, the prosthesis head has a hollow bone graft area for grafting fragments recessed on a side surface adjacent to the fractured surface. The hollow bone graft area is provided, in which fragments are grafted, so that the grafted fragments can grow together with the sclerotin of the non-fractured part, thereby reducing postoperative loosening and displacement, and then maintaining long-term efficacy.

Preferably, a recess for grafting fragments is disposed on a bottom side of the prosthesis head.

Preferably, the fragments grafted in the hollow bone graft area are fragments of a fractured bone of the radial head. The sclerotin of the fragments of the fractured part and the sclerotin of the non-fractured part are homologous, so that the two parts can better grow together. Meanwhile, it is easier to obtain the fragments of the fractured part. Thus, it is safe and reliable.

Preferably, it further comprises a prosthesis stem fixed on a bottom side of the prosthesis head, and the prosthesis stem having a shape matching with the radius below the radial head. By providing the prosthesis stem, it is convenient to operate, and the prosthesis head and the non-fractured part can be combined more firmly.

Preferably, the prosthesis stem has a plurality of first screw fixing holes. In this way, by inserting a first fixing screw into the first screw fixing holes, the prosthesis stem is fixed to the radius by the first fixing screw.

Preferably, the prosthesis further comprising two first screw fixing holes, adjacent to each other, each first screw fixing hole having a central axis formed at an angle of 10°-15° from a top surface of the non-fractured radial part; one of the first screw fixing holes is disposed at a junction between the prosthesis head and the prosthesis stem, and the other screw fixing hole is disposed at a lower end of the prosthesis stem. In this way, the prosthesis stem can be more stably fixed on the surface of the radius, and the first fixing screw can be prevented from passing through the other end of the radius, ensuring the safety of operation.

In order to enable the prosthesis head to be firmly fixed to the surface of the non-fractured part so that the fractured part and the non-fractured part are combined into a stable structure, preferably, a second screw fixing hole is disposed on the prosthesis head, and the second screw fixing hole is incommunicated with the hollow bone graft area.

Preferably, the hollow bone graft area is disposed at the lower end of the prosthesis head, and the second screw fixation holes are disposed above the hollow bone graft area.

Preferably, when in use, an included angle of 10°-15° is formed between the channel of the second screw fixing holes and the top surface of the non-fractured radial head part. In this way, a second fixing screw inserted into the second screw fixing holes can be prevented from passing through the non-fractured radial head part, ensuring the safety of operation.

Compared with the prior art, the present invention has the advantages, that the prosthesis head of the present invention is matched with the non-fractured radial head part, and can be combined with the non-fractured radial head part to form an intact radial head. In this way, when the partial articular surface of the radial head is fractured, it is unnecessary to remove the entire radial head. Instead, the radial head of the fractured part is replaced with a prosthesis head and the radial head of the non-fractured part is retained. The two parties are combined to form an intact radial head. This minimizes iatrogenic injuries to the patients, and is good for the joint intactness and joint stability.

### BRIEF DESCRIPTION OF THE DRAWINGS

FIG. 1 is a perspective view of an fracture of an entire articular surface of an radial head;

FIG. 2 is a perspective view of the fracture of the partial articular surface of the radial head;

FIG. 3 is a sectional view of a semi-radial prosthesis according to Embodiment 1 of the present invention;

FIG. 4 is a perspective view showing the semi-radial prosthesis according to Embodiment 1 of the present invention, when in use;

FIG. 5 is a sectional view of the semi-radial prosthesis according to Embodiment 2 of the present invention;

FIG. 6 is a perspective view of the semi-radial prosthesis according to Embodiment 2 of the present invention;

FIG. 7 is another perspective view of the semi-radial prosthesis according to Embodiment 2 of the present invention.

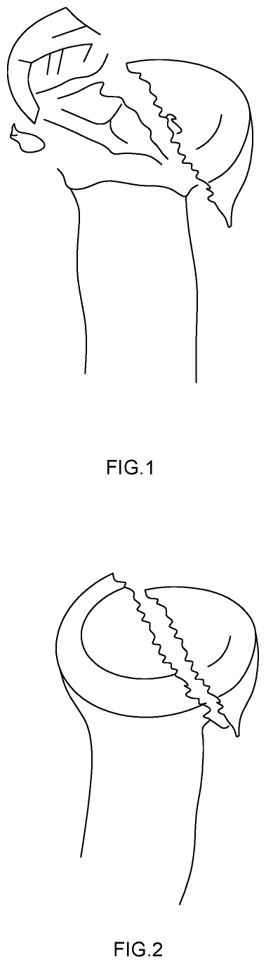

### DETAILED DESCRIPTION OF THE INVENTION

The present invention will be further described below in detail with reference to the accompanying drawings by embodiments.

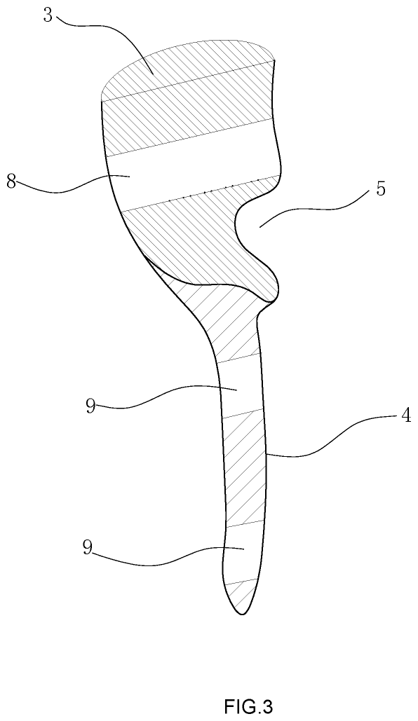

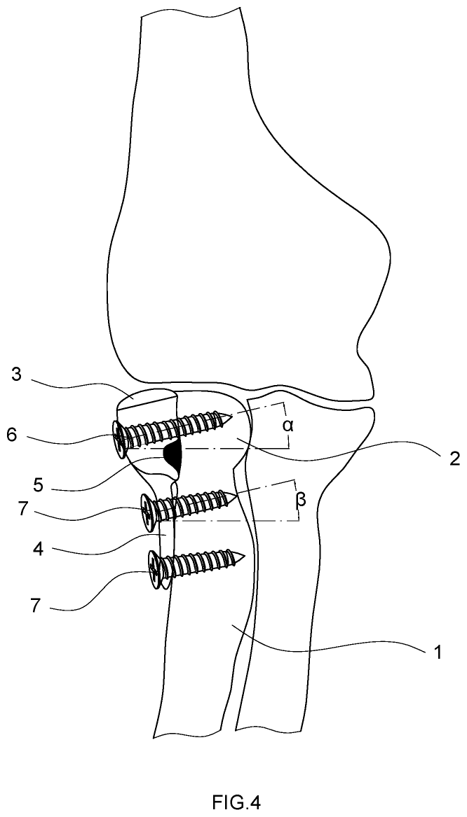

FIGS. 3 and 4 show a semi-radial head prosthesis of Embodiment 1 of the present invention. The semi-radial head prosthesis comprises a prosthesis head 3 and a prosthesis stem 4. The prosthesis stem 4 can be fixed on a bottom side of the prosthesis head 3. In this embodiment, the prosthesis head 3 and the prosthesis stem 4 are formed integratively, so that the internal structure of the semi-radial prosthesis can be more stable.

The prosthesis head 3 is matched with the non-fractured radial head part 2, and the prosthesis head 3 is jointed with the non-fractured part 2 to form an intact radial head. In this way, when the partial articular surface of the radial head is fractured, it is unnecessary to remove the intact radial head. Instead, the radial head of the fractured part is replaced with the prosthesis head 3 and the radial head of the non-fractured part is retained. The two parties are combined to form an intact radial head. This minimizes iatrogenic injuries to the patients, and is good for the joint intactness and joint stability.

The size and specification of the prosthesis head 3 are designed according to the fracture range of the patient's radial head. Preferably, the volume of the prosthesis head 3 is ¼ to ½ of the volume of the intact radial head. Thus, the prosthesis head 3 and the non-fractured radial head part form a stable combination structure. Further, in this embodiment, the prosthesis head 3 is ½ of the volume of the intact radial head. The fracture of half (about 50%) of the articular surface of the radial head is common in clinical practice. The design of the size of the prosthesis head 3 to be ½ of the intact radial head can improve the versatility of the prosthesis head 3.

In order to reduce the loosening and displacement of the prosthesis after the operation, the prosthesis head 3 has a hollow bone graft area 5 for grafting fragments recessed on a side surface adjacent to the fractured surface. The hollow bone graft area 5 is provided, in which fragments are grafted, so that the grafted fragments can grow together with the sclerotin of the non-fractured part, thereby reducing postoperative loosening and displacement, and then maintaining long-term efficacy. The fragments grafted in the hollow bone graft area 5 are fragments of a fractured bone of the radial head. The sclerotin of the fragments of the fractured part and the sclerotin of the non-fractured part are homologous, so that the two parts can better grow together. Meanwhile, it is easier to obtain the fragments of the fractured part. Thus, it is safe and reliable.

The prosthesis stem 4 has a shape matching with the radius 1 below the radial head. By providing the prosthesis stem 4, it is convenient to operate, and the prosthesis head 3 and the non-fractured part 2 can be combined more firmly. Preferably, the prosthesis stem 4 has a plurality of first screw fixing holes 9 for inserting a first fixing screw 7. In this way, by inserting the first fixing screw 7 into the first screw fixing holes 9, the prosthesis stem 4 is fixed to the radius 1 by the first fixing screw 7. In this embodiment, there are two first screw fixing holes 9 adjacent to each other. One of the first screw fixing holes 9 is disposed at a junction between the prosthesis head 3 and the prosthesis stem 4, and the other screw fixing hole 9 is disposed at a lower end of the prosthesis stem 4. In this way, the prosthesis stem 4 can be more stably fixed on the surface of the radius 1. Preferably, each first screw fixing hole having a central axis formed at an angle of 10°-15° from a top surface of the non-fractured radial part 2. In this way, the first fixing screw 7 can be prevented from passing through the other end of the radius 1, ensuring the safety of operation.

The prosthesis head 3 has a plurality of second screw fixing holes 8 for inserting a second fixing screw 6, and the second screw fixing holes 8 is incommunicated with the hollow bone graft area 5. In this way, the negative impact, caused by the screw, on the sclerotin fusion growth of the fragments in the hollow bone graft area 5 together with the non-fractured part is avoided. By inserting the second fixing screw 6 into the second screw fixing holes 8, the prosthesis head 3 can be firmly fixed to the surface of the non-fractured part, so that the fractured part and the non-fractured part can be combined into a stable structure. In this embodiment, the hollow bone graft area 5 is disposed at the lower end of the prosthesis head 3, and the second screw fixing holes 8 are disposed above the hollow bone graft area 5. In addition, when in use, an included angle of 10°-15° is formed between the channel of the second screw fixing holes 8 and the top surface of the non-fractured radial head part 2. In this way, a second fixing screw 6 inserted into the second screw fixing holes 8 can be prevented from passing through the non-fractured radial head part 2, ensuring the safety of operation.

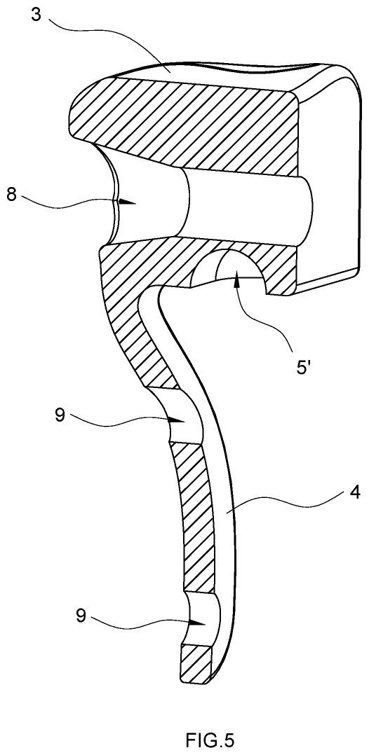

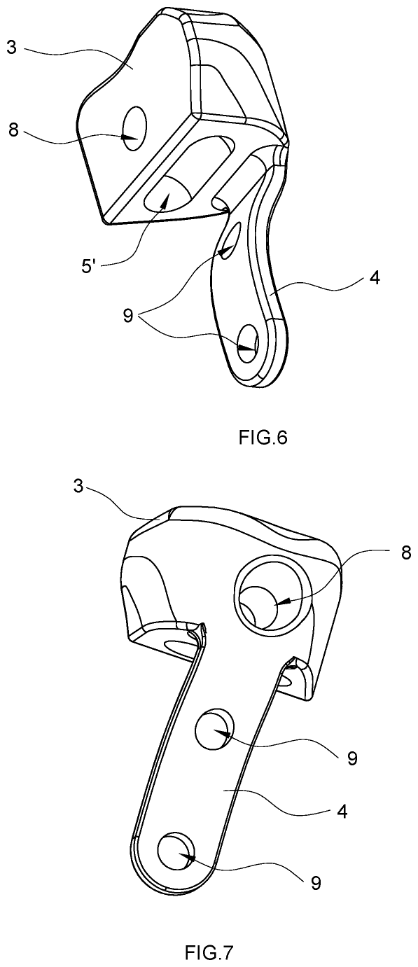

FIGS. 5 to 7 show a semi-radial head prosthesis of Embodiment 2 of the present invention. This embodiment differs from Embodiment 1 in that: a recess 5′ for grafting fragments is disposed on a bottom side of the prosthesis head 3, and the second screw fixing holes 8 is not communicated with the recess 5′. During the operation, the fragments of the fractured part can be grafted into the recess 5′.

1. A semi-radial head prosthesis for matching to a non-fractured part of a bone, comprising a prosthesis head matching to a radial head of the non-fractured part, the prosthesis head having a fractured surface, the prosthesis head is jointed with the non-fractured part to form an intact radial head.

2. The prosthesis of claim 1, wherein the volume of the prosthesis head is ½ to ¾ of the volume of the intact radial head.

3. The prosthesis of claim 1, wherein the volume of the prosthesis head is ¼ to ½ of the volume of the intact radial head.

4. The prosthesis of claim 1, wherein the prosthesis head has a hollow bone graft area for grafting fragments recessed on a side surface adjacent to the fractured surface.

5. The prosthesis of claim 1, wherein a recess for grafting fragments is disposed on a bottom side of the prosthesis head.

6. The prosthesis of claim 4, wherein the fragments grafted in the hollow bone graft area are fragments of a fractured bone of the radial head.

7. The prosthesis of claim 1, wherein further comprises a prosthesis stem fixed on a bottom side of the prosthesis head, and the prosthesis stem having a shape matching with the radius below the radial head.

8. The prosthesis of claim 7, wherein the prosthesis stem has a plurality of first screw fixing holes.

9. The prosthesis of claim 8, further comprising two first screw fixing holes, adjacent to each other, each first screw fixing hole having a central axis formed at an angle of 10°-15° from a top surface of the non-fractured radial part;
one of the first screw fixing holes is disposed at a junction between the prosthesis head and the prosthesis stem, and the other screw fixing hole is disposed at a lower end of the prosthesis stem.

10. The prosthesis of claim 4, wherein a second screw fixing hole is disposed on the prosthesis head, and the second screw fixing hole is incommunicated with the hollow bone graft area.

11. The prosthesis of claim 10, wherein the hollow bone graft area is disposed at the lower end of the prosthesis head, and the second screw fixing holes are disposed above the hollow bone graft area.

12. The prosthesis of claim 11, wherein when in use, an included angle of 10°-15° is formed between the channel of the second screw fixing holes and the top surface of the non-fractured radial head part.
